(19) United States
(12) Patent Application Publication
(10) Pub. No.: US 2009/0326657 A1
(43) Pub. Date: Dec. 31, 2009

(54) Pliable Artificial Disc Endplate

(76) Inventors: Alexander Grinberg, Newton, MA (US); Michael J. O'Neil, West Barnstable, MA (US); Andrew Dooris, Raynham, MA (US); Stephen Connolly, Sharon, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/145,724

(22) Filed: Jun. 25, 2008

(51) Int. Cl. A61F 2/44

(52) U.S. Cl. 623/17.16; 623/17.15; 623/17.11

(57) ABSTRACT

An intervertebral implant for replacing an intervertebral disc of the human spine, the implant including first and second conformable foam endplates, each endplate being conformable to a boney vertebral endplate under an anatomical load, and a core between the endplates, wherein the conformable foam endplates partition the core from the boney vertebral endplate so that the core does not contact the boney vertebral endplate.

### BACKGROUND OF THE INVENTION

A human intervertebral disc has several important functions, including functioning as a spacer, a shock absorber, and a motion unit. In particular, the disc maintains the separation distance between adjacent boney vertebral bodies. The separation distance allows motion to occur, with the cumulative effect of each spinal segment yielding the total range of motion of the spine in several directions. Proper spacing is important because it allows the intervertebral foramen to maintain its height, which allows the segmental nerve roots room to exit each spinal level without compression. Further, the disc allows the spine to compress and rebound when the spine is axially loaded during such activities as jumping and running. Importantly, it also resists the downward pull of gravity on the head and trunk during prolonged sitting and standing. Furthermore, the disc allows the spinal segment to flex, rotate, and bend to the side, all at the same time during a particular activity. This would be impossible if each spinal segment were locked into a single axis of motion.

An unhealthy disc may result in pain. One way a disc may become unhealthy is when the inner nucleus dehydrates. This results in a narrowing of the disc space and fibers can crack and tear. Further, loss of normal soft tissue tension may allow for a partial dislocation of the joint, leading to bone spurs, foraminal narrowing, mechanical instability, and pain.

Lumbar disc disease can cause pain and other symptoms in at least two ways. First, if the annular fibers stretch or rupture, the nuclear material may bulge or herniate and compress neural tissues resulting in leg pain and weakness. This condition is often referred to as a pinched nerve, slipped disc, or herniated disc. This condition typically will cause sciatica or radiating leg pain, as a result of mechanical and/or chemical irritation against the nerve root. Although the overwhelming majority of patients with a herniated disc and sciatica heal without surgery, if surgery is indicated it is generally a decompressive removal of the portion of herniated disc material, such as a discectomy or microdiscectomy.

Second, mechanical dysfunction can cause disc degeneration and pain (e.g. degenerative disc disease). For example, the disc may be damaged as the result of some trauma that overloads the capacity of the disc to withstand increased forces passing through it, and inner or outer portions of the annular fibers may tear. These torn fibers may be the focus for inflammatory response when they are subjected to increased stress, and may cause pain directly, or through the compensatory protective spasm of the deep paraspinal muscles.

Traditionally, spinal fusion surgery has been the treatment of choice for individuals who have not found pain relief for chronic back pain through conservative treatment (such as physical therapy, medication, manual manipulation, etc), and have remained disabled from their occupation, from their activities of daily living, or simply from enjoying a relatively pain-free day-to-day existence. There have been significant advances in spinal fusion devices and surgical techniques. However, the procedures generally include shaping two adjacent boney vertebral endplates to conform to the endplates of the fusion device. The removal of bone from the endplates weakens the vertebral bodies and can lead to device stress shielding, bone remodeling, device subsidence, and device expulsion or migration. Further, known endplates can lead to uneven distribution of loads across the vertebral bodies.

Known artificial discs offers several theoretical benefits over spinal fusion for chronic back pain, including pain reduction and a potential to avoid premature degeneration at adjacent levels of the spine by maintaining normal spinal motion. However, like spinal fusion surgery, the removal of bone from the vertebral endplates typically is necessary, thereby, weakening the vertebral bodies. Further, known endplates cause uneven distribution of loads across the vertebral bodies(?).

Therefore, a need exists for an intervertebral implant and a method replacing an artificial disc that overcomes or minimizes the above-referenced problems.

US Published Patent Application No. 2007/0225811 (Scifert) discloses compound orthopedic implants, intervertebral prosthetic implants and methods of treating a patient. In an exemplary embodiment, a compound orthopedic implant comprises a first conformable body and a second conformable body overlying the first conformable body. The compound orthopedic implant can function as a conformable carrier for delivering a therapeutic agent to an orthopedic site.

US Published Patent Application No. 2006/0282166 (Molz) discloses intervertebral implant components having compliant coatings, and methods of making and implanting the implant components. The embodiments relate to compositions, methods and devices having a compliant surface coating that permits application of the device in areas without significant bone reformation to accept the device.

US Published Patent Application No. 2006/0111785 (O'Neil) discloses an intervertebral implant replaces an intervertebral disc of the human spine. The intervertebral implant includes a first conformable endplate, the first conformable endplate being conformable to a boney vertebral endplate under an anatomical load, a second endplate and a core between the endplates, wherein the first conformable endplate partitions the core from the boney vertebral endplate, whereby the core does not contact the boney vertebral endplate. The invention is also directed to a method of replacing an intervertebral disc.

US Published Patent Application No. 2004/0010318 (Ferree) discloses an anatomical artificial disc replacement (ADR) device includes a tray having a surface which is convex to better conform to a concavity in a vertebral endplate. In different preferred embodiments, the tray may be constructed of multiple pieces adapted to conform to the vertebral endplate; a flexible material such as a malleable metal to fit the vertebral endplate; or a substrate and an attachable convex piece configured to conform to the concavity. Alternatively, the tray includes a substrate and an injectable material that hardens in situ to fill the concavity. The injectable material may be a liquid metal or a polymer, and may be injected along diverging or converging paths to minimize pull-out.

US Published Patent Application No. 2003/0069642 (Ralph) discloses an artificial intervertebral disc having a pair of opposing plate members for seating against opposing vertebral bone surfaces, separated by a spring mechanism. The preferred spring mechanism is at least one spirally slotted belleville washer having radially extending grooves.

### SUMMARY OF THE INVENTION

The invention is generally related to an intervertebral implant for replacing at least a portion of an intervertebral disc of the human spine. The intervertebral implant includes a first conformable foam prosthetic endplate, the first conformable foam prosthetic endplate being conformable to a boney vertebral endplate under an anatomical load, a second prosthetic endplate, and a core between the endplates, wherein the first conformable foam prosthetic endplate partitions the core from the boney vertebral endplates, whereby the core does not contact the boney vertebral endplate. The implant can be an artificial disc or a fusion cage.

In one embodiment of the invention, the second prosthetic endplate is also foam and is conformable to a second boney vertebral endplate under an anatomical load. Further, the second conformable foam prosthetic endplate partitions the core from the second boney vertebral endplate, whereby the core does not contact the second boney vertebral endplate.

In one embodiment of the invention, at least one rigid plate can be disposed between at least one of the first and second comformable foam endplates and the core, the rigid plate including a material which does not deform under the anatomical load.

The comformable foam endplate of the present invention can be made from at least one material selected from the group consisting of a metallic, a polymeric, and a ceramic. The metallic material includes at least one member selected from the group consisting of titanium, tantalum, cobalt-chromium, nitinol, and stainless steel. The polymeric material includes at least one member selected from the group consisting of polyethylene, polyester, polyurethane, silicone, and polycarbonate. The ceramic material includes at least one member selected from the group consisting of zirconia, alumina, hydroxyapatite, and tricalcium phosphate.

At least one protrusion element can be optionally coupled to a surface of at least one of the first and second endplates, the protrusion element being capable of penetrating a boney vertebral endplate, thereby securing a position of the first or second endplate to the boney vertebral endplate. The protrusion element includes at least one member selected from the group consisting of a keel, a spike, a tooth, a fin, and a peg.

In one embodiment of the invention, the core is between the endplates, the core supporting boney vertebral endplates between which the conformable endplates have been placed and wherein the position of each conformable endplate is controlled at least in part by the boney vertebral endplate to which it is attached and is independent of the position of the other endplate. Optionally, the core can be a non-fluid or the core can include an osteoinductive rigid matrix which provides for spinal fusion.

In one embodiment of the invention, a kit includes at least two first conformable foam endplates. Each first conformable foam endplate of the kit is conformable to a boney vertebral endplate under an anatomical load. Each first conformable foam endplate has at least one dimension that is distinct from another first conformable foam endplate of the kit. Each second endplate has at least one dimension that is distinct from another second endplate of the kit. A core is dimensioned for implantation between a first conformable foam endplate and a second endplate in an intervertebral space that has been prepared for placement of the first conformable foam endplate, the second endplate and the core. Upon implantation, the first conformable foam endplate partitions the core from a first boney vertebral endplate with which the first conformable foam endplate is in contact, whereby the core does not contact the first boney vertebral endplate.

In one embodiment of the invention, the second conformable foam prosthetic endplate is conformable to a second boney vertebral endplate under an anatomical load. Further, upon implantation of the second conformable foam prosthetic endplate and the core into an intervertebral space that has been prepared for placement of the first conformable endplate, the core and the second conformable foam prosthetic endplate, the second conformable foam prosthetic endplate partitions the core from the second boney vertebral endplate, whereby the core does not contact the second boney vertebral endplate.

In one embodiment of the invention, an intervertebral implant includes two conformable foam prosthetic endplates. Each conformable foam prosthetic endplate includes a foam material that conforms to a boney vertebral endplate under an anatomical load and a core between the endplates. The core supports boney vertebral endplates between which the conformable foam prosthetic endplates have been placed. The position of each conformable foam prosthetic endplate is controlled at least in part by the boney vertebral endplate to which it is attached and is independent of the position of the other foam prosthetic endplate.

The invention is also directed to a method of replacing an intervertebral disc. The method includes removing at least a portion of an intervertebral disc to form an intervertebral disc space, implanting a first conformable foam prosthetic endplate into the intervertebral disc space and in contact with a first boney vertebral endplate. The first conformable foam prosthetic endplate is conformable to the first boney vertebral endplate under an anatomical load. A second endplate is implanted into the intervertebral disc space and is in contact with a second boney vertebral endplate. A core is implanted between the first conformable foam prosthetic endplate and the second endplate, wherein the first conformable foam prosthetic endplate partitions the core from the first boney vertebral endplate. The core does not contact the first boney vertebral endplate.

In one embodiment of the method of the invention, the second conformable endplate is conformable to the second vertebral endplate under an anatomical load. Further, upon implantation, the second conformable endplate partitions the core from the second boney vertebral endplate, whereby the core does not contact the second boney vertebral endplate.

In one embodiment of the method of the invention, at least one rigid plate can be implanted between the core and at least one of the first conformable foam endplate and the second endplate.

The invention has many advantages. For example, the invention provides the boney vertebral bodies from succumbing to device stress shielding, bone remodeling, device subsidence, and device expulsion. Further, the invention also allows for even load distribution across the boney vertebral bodies.

### BRIEF DESCRIPTION OF THE DRAWINGS

FIG. 1 shows a perspective view of the lower region of the spine in which an intervertebral space has been prepared for implantation of the invention.

FIG. 2 shows a perspective view of one embodiment of the artificial implant of the invention being inserted into the prepared intervertebral space of the lumbar spine region of FIG. 1.

FIG. 3A shows an exploded perspective view of on embodiment of the implant of the present invention.

FIG. 3B shows an exploded perspective view of the implant of FIG. 3A with securing elements attached.

FIG. 3C shows an exploded perspective view of another embodiment of the present invention;

FIG. 3D shows an exploded perspective view of the implant of FIG. 3C with securing elements attached.

FIG. 4A shows a view of another embodiment of the present invention highlighting movement of spine in relation to the invention.

FIG. 4B shows another view of FIG. 4A.

FIG. 4C shows another view of FIG. 4A.

FIG. 5 shows a view of a prior art embodiment highlighting movement of spine about a pivot point.

FIG. 6 is a cross section of a comformable foam endplate of the present invention comprising a high density perimeter section and a low density central section.

FIGS. 7a and 7b disclose a prosthetic endplate having a dispensing inlet located on a sidewall, the inlet fluidly connected to intra-endplate channel, which is fluidly connected to the dispensing orifice located on an outer surface of the prosthetic endplate.

FIG. 8 discloses an endplate of the present invention attached to a bony endplate via injected cement.

### DETAILED DESCRIPTION OF THE INVENTION

The foregoing and other objects, features and advantages of the invention will be apparent from the following more particular description of preferred embodiments of the invention, as illustrated in the accompanying drawings in which like reference characters refer to the same parts throughout the different views. The same number appearing in different drawings represent the same item. The drawings are not necessarily to scale, emphasis instead being placed upon illustrating the principles of the invention.

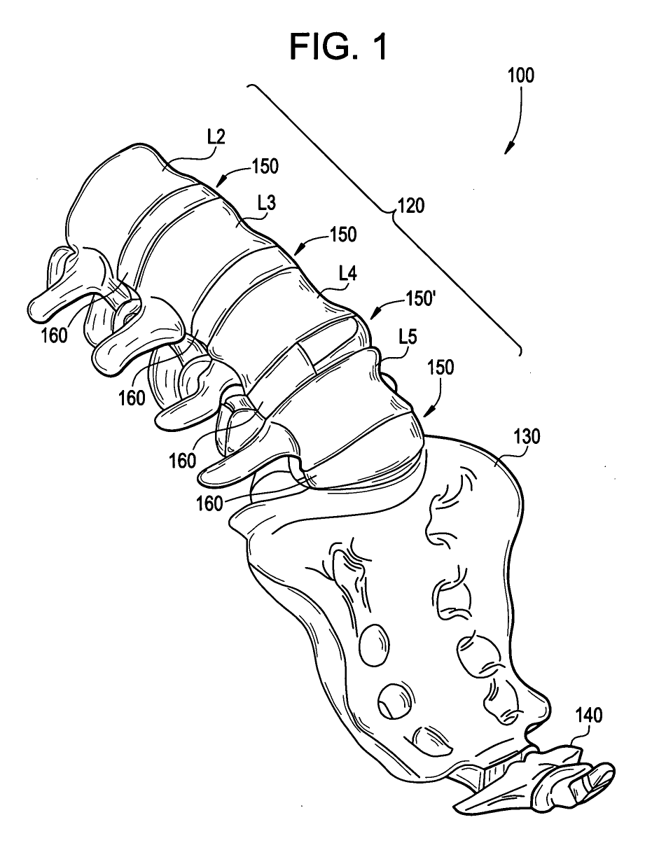

The present invention is related to a conformable implant intended to replace an intervertebral disc which has been removed due to disease, infection, deformity, or fracture, for example. FIG. 1 shows a perspective view of the lower region of a human spine 100. This region includes lumbar spine 120, sacral spine 130, and coccyx 140. The lumbar spine 120 is comprised of five (5) vertebrae L5, L4, L3, L2, and L1 (not shown). Intervertebral discs 150 link contiguous vertebra from C2 (not shown) to the sacral spine 130, wherein a single apostrophe (') denotes a damaged disc, such as 150′.

Intervertebral disc 150 includes a gelatinous central portion called the nucleus pulposus (not shown) which is surrounded by an outer ligamentous ring called annulus fibrosus 160. The nucleus pulposus is composed of 80-90% water. The solid portion of the nucleus is Type II collagen and non-aggregated proteoglycans. Annulus fibrosus 160 hydraulically seals the nucleus pulposus, and allows intradiscal pressures to rise as the disc is loaded. Annulus fibrosus 160 has overlapping radial bands which allow torsional stresses to be distributed through the annulus under normal loading without rupture.

Annulus fibrosus 160 interacts with the nucleus pulposus. As the nucleus pulposus is pressurized, the annular fibers of the annulus fibrous prevent the nucleus from bulging or herniating. The gelatinous material directs the forces of axial loading outward, and the annular fibers help distribute that force without injury.

Although the following procedure is explained with reference to the lower spine, the procedure can be performed on any damaged disc of the spine. Further, the following procedure is described with reference to implants. However, it should be understood by one skilled in the art that an implant may be an artificial disc, a spinal fusion cage, or any other device known in the art.

According to one embodiment of the method of the invention, damaged disc 150′ is prepared to receive an implant of the invention by removing a window the width of the implant to be implanted from the annulus 160 of the damaged disc 150′. The nucleus pulposus of the disc 150′ is removed.

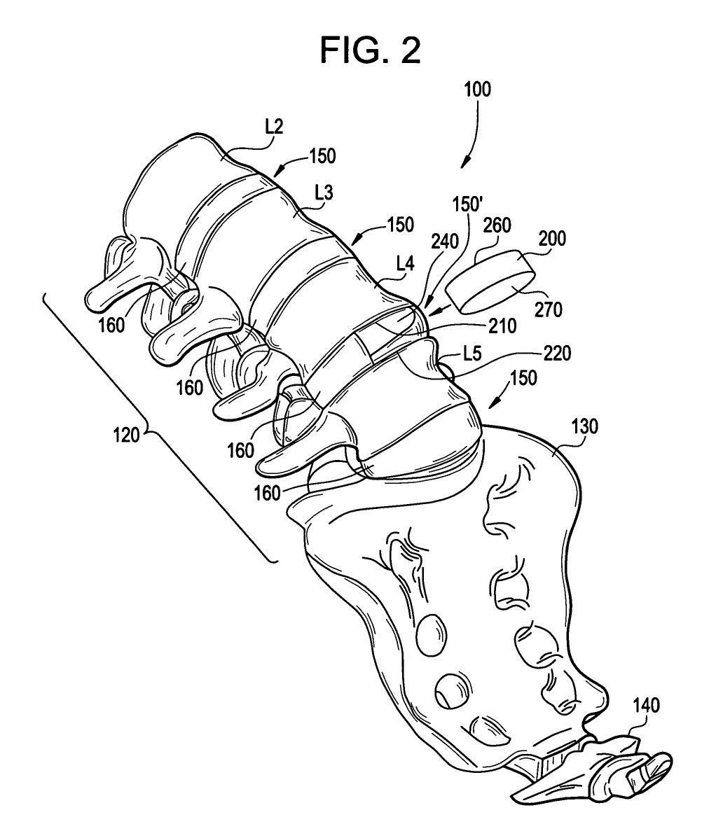

Referring to FIG. 2, once the damaged disc space is prepared, the surgeon chooses implant 200 of the invention from a kit of implants (not shown). The kit contains prefabricated and modular implants of various heights, shapes, and sizes. The surgeon inserts the chosen implant 200 into the intervertebral space 210 located between the superior endplate 220 of the inferior vertebra L5 and the inferior endplate 240 of the superior vertebra L4 (each vertebral body has a superior endplate and an inferior endplate).

The implant 200 may be inserted by hand or with an insertion instrument (not shown). If the implant 200 does not closely match the intervertebral space 210, the surgeon removes the implant 200 and chooses another implant 200 from the kit. This step is repeated until the surgeon determines the implant 200 which closely matches the intervertebral space 210.

The surgeon may then adjust the position of the implant 200 in the intervertebral space if needed. The implant can be adjusted in any direction within the intervertebral space 210. The implant 200 is now ready to be secured to vertebral endplates.

In one embodiment, either superior endplate 260 or inferior endplate 270 of the implant 200 conform to the defined contours (i.e. shapes) of superior or inferior endplates 220,240, respectively of vertebral bodies under an anatomical load. Preferably, both superior endplate 260 and inferior endplate 270 conform to boney vertebral endplates with which they are in contact.

In another embodiment one endplate is conformable and the other one is rigid based on the patient anatomy or bone conditions, providing the surgeon with a choice of options.

If one endplate is not conformable to the boney vertebral endplate with which it is in contact, then that endplate can be a rigid material that is suitable for implantation, such as a rigid bio-compatible, metallic, polymeric or biologic material. In the embodiment wherein the second endplate is rigid, the method of the invention can, optionally, include a step of preparing a portion of a second boney vertebral endplate for implantation of the second endplate, such as by grinding or cutting the second boney vertebral endplate. The anatomical load is the weight of the body above the resulting disc space, i.e., the weight of the body above disc space 210 in FIG. 2. In prior art techniques, the superior and inferior endplates of vertebral bodies were shaped to conform to the implant endplates.

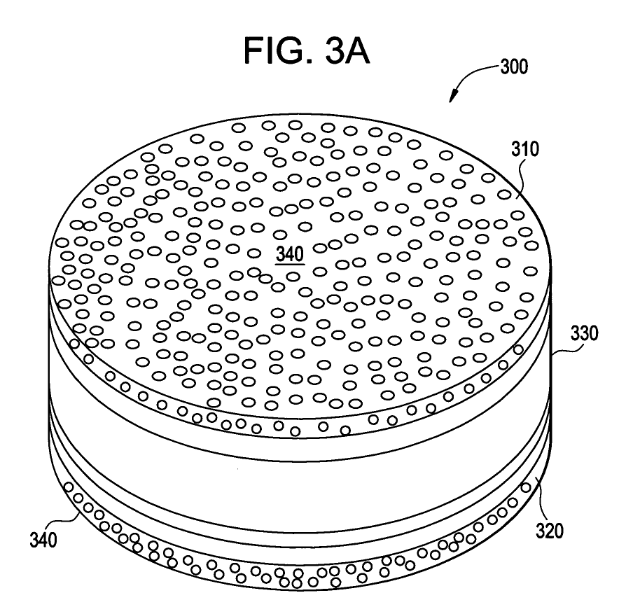

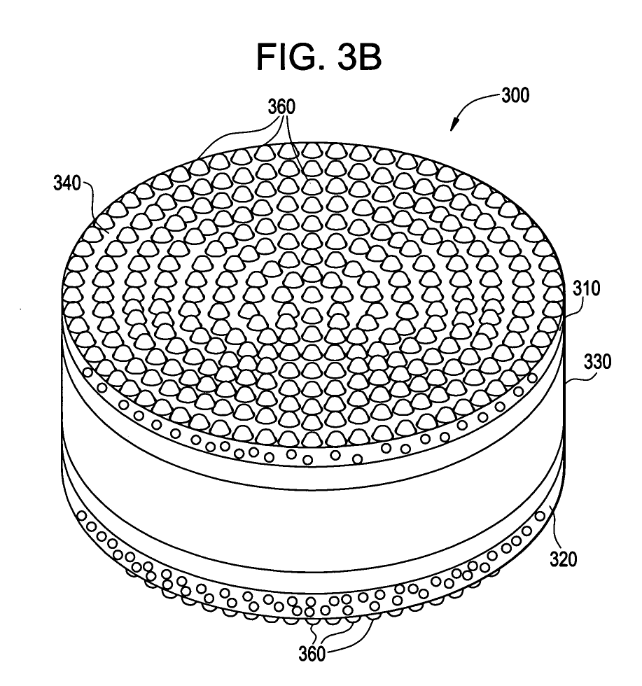

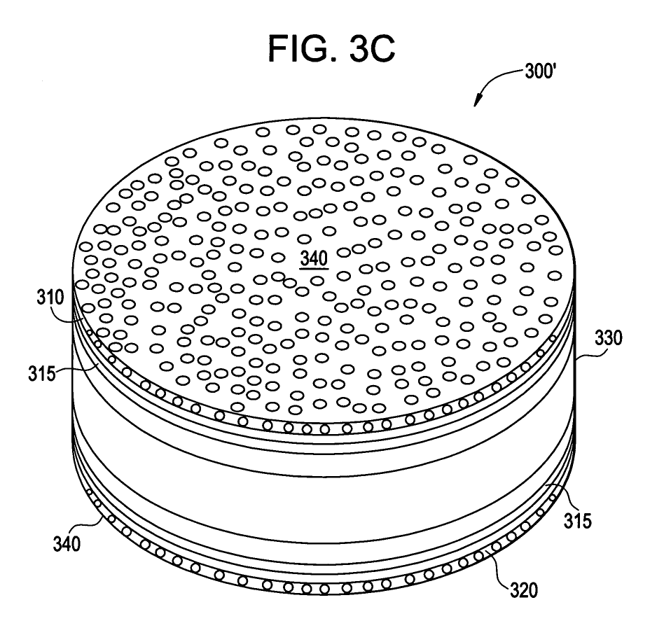

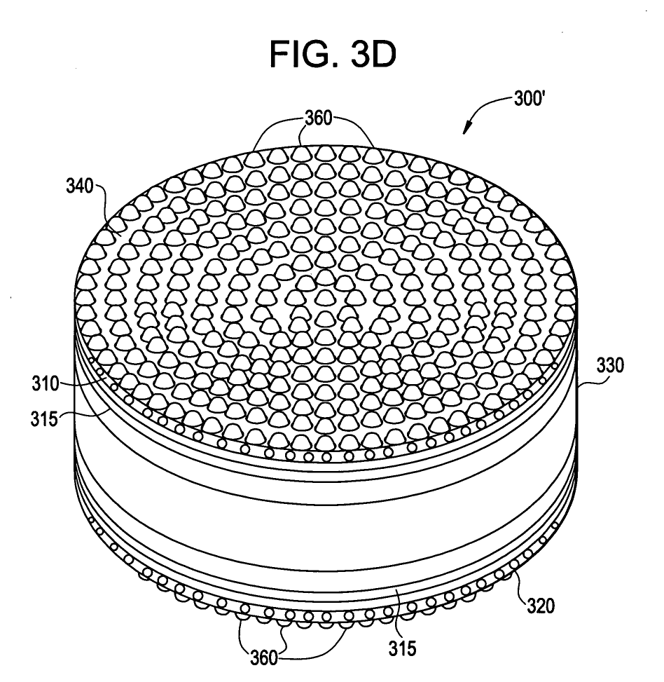

The implant 200 can be further secured to the vertebral bodies by attaching at least one protrusion element (360 FIGS. 3B and 3D) to the superior and inferior endplates 260, 270 of the implant 200 to secure the implant 200 to vertebral endplates 220,240. The protrusion element 360 can be a keel, a spike, a tooth, a fin, or a peg.

FIGS. 3A and 3B show exploded views of a conformable implant 300 of an embodiment of the present invention and FIGS. 3C-3D show exploded views of a conformable implant 300′ of another embodiment of the present invention.

Each implant 300, 300′ has superior conformable foam endplate 310, inferior conformable foam endplate 320, and core 330 disposed between the superior endplate 310 and the inferior endplate 320. Each foam endplate 310,320 has an endplate surface 340 that is entirely conformable which allows for even load distribution across the boney vertebral bodies. Each foam endplate 310,320 also partitions the core from boney vertebral endplates contacting surface 340 of each foam endplate 310,320, whereby the core does not contact the boney vertebral endplates. However, in the embodiment of FIGS. 3C and 3D, a rigid plate 315 which does not deform under an anatomical load can be disposed between each foam endplate 310,320 and the core 330. It should be understood that a single rigid plate or multiple rigid plates can be used in any combination desired by the surgeon. For example, the surgeon may choose an implant 300′ with three rigid plates 315 disposed between the superior foam endplate 310 and the core 330 while having no rigid endplates 315 between the inferior foam endplate 320 and the core 330.

The comfortable foam endplate of the present invention can be made from at least one member selected from the group consisting of a metallic, polymeric, or ceramic material or any combination thereof which conforms to the boney vertebral endplate upon anatomical loading. Examples of these materials include, but are not limited to, titanium, tantalum, cobalt-chromium, stainless steel, nitinol, polyethylene, polyester, polyurethane, silicone, polycarbonate, or other flexible materials which exceed the yield limit following loading which allows the endplate to conform.

Preferably, the conformable foam endplate is specifically designed to flex in a controlled fashion by manipulating its thickness, pore size, density and depth.

In one embodiment, titanium foam produced commercially by Stealth Medical Technologies, 199 South Mount Pleasant Rd., Collierville, Tenn. 38017, is used as the endplate material of construction. The pliable endplate is preferably made from titanium foam but the foam can also be produced from other metallic materials including cobalt—chrome and stainless steel. The density and porosity of the titanium foam can be controlled to allow for more or less conformance to the vertebral body.

In some embodiments, the metallic foam endplate is made in substantial accordance with US Patent Publication 2002-0120336, the specification of which is incorporated by reference in its entirety. In one technique, the endplate is made of foam metal that contains a plurality of interconnected voids. Foam metal, produced by mixing a powdered foaming agent with a metal powder, is a porous metal matrix with unique properties. One technique for forming foam metal is commonly known as “free-foaming.” During free-foaming, a billet of metal containing a foaming agent is placed in a furnace and is heated to temperatures greater than the matrix metal. As the billet melts, the foaming agent releases gas in a controlled way. The gas discharge slowly expands the metal as a semi-solid foamy mass. The foaming process stops as the metal cools. Density is controlled by changing the foaming agent content and varying the heating conditions. U.S. Pat. No. 5,151,246, the disclosure of which is incorporated herein by reference in its entirety, discloses a suitable technique for the manufacture of foam metal that could be used to produce the endplate of the present invention. Another technique for forming foam metal is to mix a small quantity of powdered foaming agent with conventional metal powders to form a billet. The billet is heated by induction coils to a foaming temperature. The now-liquid billet is injected in a foaming state into complex molds. The injection of molten foam provides a versatile way to produce complex shapes of foam metal and can be utilized to produce an endplate with non-uniform geometries.

In some embodiments, the metallic foam endplate is made in substantial accordance with US Patent Publication 2005-0048193 the specification of which is incorporated by reference in its entirety. This involves preparing porous bodies, from which metal articles can be made, by the so-called slip casting process. The slip casting process comprises the preparation of a body by the impregnation of a pyrolysable foam material, such as a polymer, with a slurry of metal particles, and subsequent pyrolysis of the foam material. This may subsequently be followed by sintering of the body. Therefore, in some embodiments, the present invention is directed to a method for preparing a porous body, suitable for the production of a porous metal article, comprising the steps of providing a polymeric foam, which foam is impregnated with a slurry of metal particles, drying the impregnated foam, followed by pyrolysis in the presence of metal hydride particles. Using this process, it is possible to produce endplates that have a porous metal structure with a porosity of at least 50%, having a mean pore size of at least 400 microns, wherein the pores are interconnected. The porous metal endplates of the invention have a compressive strength ranging from 5 MPa up to 40 MPa, or even higher. Strength is obviously related to porosity. In the case of 80% porous titanium alloy, a compressive strength of 10 MPa or higher may be obtained in accordance with the invention, which is suitable for applications in implants. Typically, 50-90% porous endplates can be provided, having a compressive strength ranging from 5-40 MPa. The mechanical compressive strength which may be obtained in accordance with the present invention is sufficient for load-bearing purposes.

The thickness of the material varies depending upon the ductility of the material used, for example, titanium 64 can range between 0.0625 mm to 1 mm in thickness, whereas commercially pure titanium can range between 0.0625 mm to 6.35 inches in thickness. The plate thickness could range from 0.3 mm for cervical disc and to about 1 mm for lumbar disc. The porous structure may be manufactured through the thickness of the plate or just deep enough to allow bone ingrowth. The physical properties of Ti plate providing a rigid support to the axial spine loads without collapsing, but the plate will flex due to small thickness to accommodate the vertebra endplate geometry.

The endplates could be attached to the disc core by different methods like insert molding, gluing, vulcanization and other methods.

In another embodiment the core could be made from a hard material and the endplate from a softer material (polyurethanes and other similar materials). The endplate will act as a shock absorber and will conform to the shape of the vertebra. It could be attached in a similar fashion as described above for Ti Foam and the pliable core.

Various methods known in the art can be employed singularly or in combination to help facilitate bone growth into the foam endplate. For example, each foam endplate 310,320 can include endplate surface 340 that is textured or roughened, whereby conformable foam endplate 310,320 bind to boney vertebral endplates upon boney ingrowth of the boney vertebral endplates into textured endplate surface 340 of each foam endplate 310,320. Examples of a textured or roughened endplate surface include porous beading, hydroxyapatite, and mesh. Further, endplate surface 340 of each foam endplate 310,320 can be coated with an osteoinductive or osteoconductive material. Osteoconductive materials can be porous metallic, polymeric, ceramic, or biologic materials or any combination thereof. Examples of osteoinductive materials include, but are not limited to, bone morphogenic proteins, demineralized bone matrices, growth factors or other materials known to facilitate bone growth.

The top surface facing the bone could be coated with hydroxyapatite to promote bone ingrowth. The rough surface of the plate will provide stability to the implant in the disc space.

Protrusion elements 360 can also be attached to the endplate surfaces 340 to provide against disc expulsion. Examples of protrusion elements include keels, spikes, teeth, fins, and pegs.

The core 330 of the implant 300,300′ can provide relative movement of the foam endplates 310,320 about the spine, such as a core in an artificial disc. An example of one such core is described in U.S. Pat. No. 5,401,269, and another example is described in U.S. Provisional Application No. 60/391,845, filed Jun. 27, 2002, the entire teachings of which are incorporated herein by reference.

Although a main part of this invention relates to the spinal artificial discs, it can also be utilized in other areas of the human anatomy where a good opposition between the implant and the bone or tissue is important. Therefore, alternatively, as is the case with a fusion cage, the core 330 of the implant 300,300′ can be made from an osteoinductive rigid matrix or cage with struts that are inter-packed with bone to provide short term rigidity and provide for long term ingrowth.

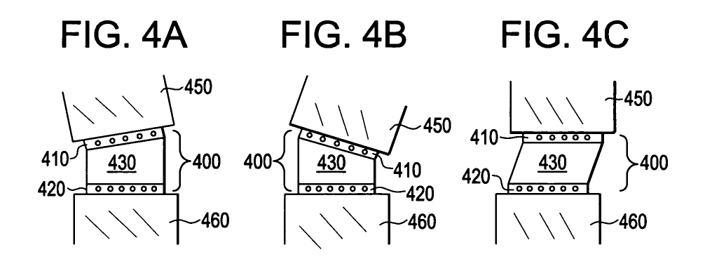

Referring to FIGS. 4A-4C, in another embodiment of the invention the implant 400 does not have a fixed pivot point within core 430. Each foam endplate 410,420 of implant 400 moves independent of each other, that is, each foam endplate 410,420 moves relative to its adjacent boney vertebra 450,460. The ability for implant 400 not to have a fixed pivot point allows the implant mimic a normal intervertebral disc of the spine. In contrast, prior art implants 500 as shown in FIG. 5 and described in more detail in U.S. Patent Publication 2003/0069642, the entire teachings of which are incorporated herein by reference, have a fixed pivot point 540 at the center of core 530 which does not allow for independent movement of endplates 510,520 relative to its adjacent boney vertebra 550,560.

In some motion disc embodiments, the artificial disc core is flexible and is preferably selected from conformable materials (such as a polyurethane or a silicone) that provide axial resistance to support the spine but at the same time accommodate flexion/extension, lateral bending and axial rotation. More preferably, the endplates of such a motion disc are thin plates made of titanium foam.

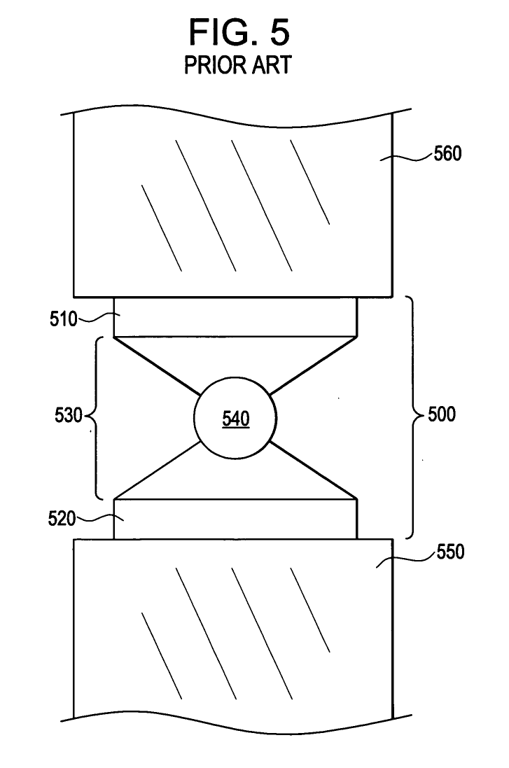

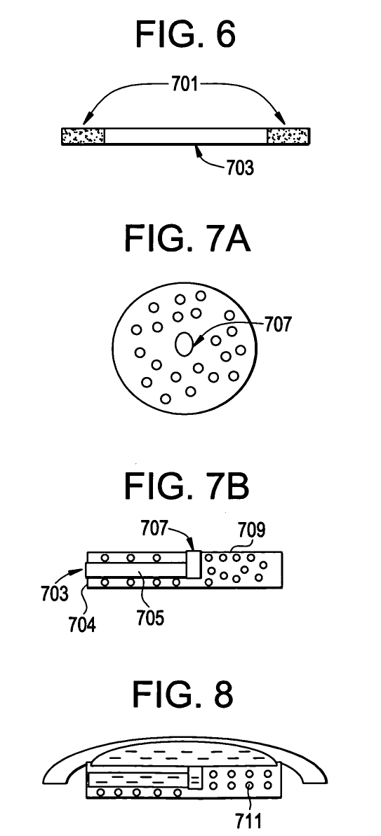

Now referring to FIG. 6, specific features of the endplate can be comprised of thicker or thinner layers and/or various densities to control the degree of boney conformance as well as boney interdigitation. For example, in FIG. 6, the endplate comprises a high density edge section 701 and a low density central section 703. The advantage of this design is that the edge section is located in the relatively flat and hard (cancellous bone) area of the vertebra and provides and adequate axial support. The central area of the plate is more comformable due to less density and located in concave area of the vertebra where more flexibility is needed to conform to the vertebra.

The endplate could have a pocket at its inner surface for core insertion. In this embodiment, the different shapes of the endplates could be assembled to the core during surgery based on patient needs.

Now referring to FIGS. 7a and 7b, another embodiment of the invention is injecting a bone cement or bone ingrowth agent through the artificial disc or vertebra as a filler to close any remaining nonconformity of the endplate with the vertebra, or to help the bone to adhere to the endplate of the artificial disc. For injecting cement through the disc, the flexible core could have channels on the top and bottom surfaces connected to the center hole of the plates. If injecting through the vertebra, the hole has to be drilled on an angle through the vertebra or the needle has to be pushed between the endplate and the vertebra. For example, in FIGS. 7a and 7b, the prosthetic endplate has a dispensing inlet 703 located on a sidewall 704, the inlet fluidly connected to intra-endplate channel 705, which is fluidly connected to the dispensing orifice 707 located on an outer surface 709 of the prosthetic endplate. In this way, once the prosthetic endplate is seated in the bony endplate, osteoconductive injectate can be injected into the dispensing inlet, travel through the channel and exit the dispensing orifice in order to fill a) the porosity of the porous prosthetic endplate and b) the gap between the prosthetic endplate and the bony endplate, thereby providing a tight grip between the prosthetic and bony endplates.

FIG. 8 discloses an endplate 711 of FIGS. 7a-7b attached to a bony endplate via injected cement.

Another embodiment is designing and machining or molding the pliable endplates from CT scan images of the vertebra endplate that is precisely machined to mirror the patient's anatomy. The endplate produced in this way could be attached to the core intraoperatively or preassembled to the core before surgery at the factory. This method provides a custom-made implant for a particular patient. Another embodiment has a set of endplates of different configurations—thicknesses, shapes, sizes, stiffness, and porosities—which can be assembled to the core in the operating room after determining the best match for the patient's anatomy.

In addition to the above embodiments, a trial device incorporating conformable endplates can also be made by using permanently deformable materials, such as urethane foam. This trial device may be inserted into the disc space, pressed to the endplate and then removed. The trial will allow the surgeon to obtain a 3-dimensional representation of the disc space, properly prepare the surface of the endplate, and then select the implant accordingly.

In another embodiment, the Ti or CoCr beads (irregular shapes) are molded into or imbedded into the surface of the disc pliable core itself providing the strength, hardness and roughness to the core top and bottom surfaces. This allows the device designer to eliminate endplates all together.

In some embodiments, the conformable foam endplate comprises attachments thereto.

In some embodiments, the core is inflexible and the conformable endplate comprise a rubber-like material.

In some embodiments, the conformable foam endplate is custom made from a CT scan.

The above discussion has used an anterior surgical approach to the spine. However, other approaches such as posterior, lateral and posterolateral approaches may be used as well.

The present invention also contemplates a trial with a conformable endplate. Thus, in some embodiments, there is provided an intervertebral implant trial, comprising:
(a) a first conformable foam prosthetic endplate conformable to a first boney vertebral endplate under an anatomical load;
(b) a second prosthetic endplate; and
(c) a core between the prosthetic endplates.
   Also, in some embodiments, there is provided an intervertebral implant, comprising:
(a) a comformable core having an upper surface and a lower surface,
(b) a first plurality of beads embedded in the upper surface of the core, and
(c) a second plurality of beads embedded in the lower surface of the core.
   While this invention has been particularly shown and described with references to preferred embodiments thereof, it will be understood by those skilled in the art that various changes in form and details may be made therein without departing from the scope of the invention encompassed by the appended claims.

We claim:

1. An intervertebral implant, comprising: (a) a first conformable foam prosthetic endplate conformable to a first boney vertebral endplate under an anatomical load; (b) a second prosthetic endplate; and (c) a core between the prosthetic endplates, wherein the first conformable foam prosthetic endplate partitions the core from the boney vertebral endplate, whereby the core does not contact the boney vertebral endplate.

2. The intervertebral implant of claim 1, wherein the second endplate is a conformable foam endplate that is conformable to a second boney vertebral endplate under an anatomical load.

3. The intervertebral implant of claim 2, wherein the second endplate partitions the core from the second boney vertebral endplate, whereby the core does not contact the second boney vertebral endplate.

4. The intervertebral implant of claim 1, further including at least one rigid plate disposed between at least one of the first and second endplates and the core, the rigid plate including a material which does not deform under the anatomical load.

5. The intervertebral implant of claim 1, wherein both the first and second endplate includes a textured surface that facilitates bone growth.

6. The intervertebral implant of claim 5, wherein the textured surface is treated with an osteoinductive or osteoconductive material.

7. The intervertebral implant of claim 1, wherein the core is flexible.

8. The intervertebral implant of claim 7, wherein the osteoinductive or osteoconductive material includes at least one member selected from the group consisting of a metallic, a polymeric, a ceramic, and a biologic material.

9. The intervertebral implant of claim 8, wherein the metallic material includes at least one member selected from the group consisting of titanium, tantalum, cobalt-chromium, nitinol, and stainless steel.

10. The intervertebral implant of claim 8, wherein the polymeric material includes at least one member selected from the group consisting of polyethylene, polyester, polyurethane, silicone, and polycarbonate.

11. The intervertebral implant of claim 8, wherein the ceramic material includes at least one member selected from the group consisting of zirconia, alumina, hydroxyapatite, and tricalcium phosphate.

12. The intervertebral implant of claim 8, wherein the biologic material includes at least one member selected from the group consisting of collagen, bone morphogenic protein, a demineralized bone matrix, and a growth factor.

13. The intervertebral implant of claim 1, further including at least one protrusion element coupled to a surface of at least one of the first and second endplates, the protrusion element being capable of penetrating a boney vertebral endplate, thereby securing a position of the first or second endplate to the boney vertebral endplate.

14. The intervertebral implant of claim 13, wherein the protrusion element includes at least one member selected from the group consisting of a keel, a spike, a tooth, a fin, and a peg.

15. The intervertebral implant of claim 1, wherein the conformable material includes at least one member selected from the group consisting of a metallic, a polymeric, and a biologic material.

16. The intervertebral implant of claim 1, wherein the core between the endplates, the core supporting boney vertebral endplates between which the conformable endplates have been placed and wherein the position of each conformable endplate is controlled at least in part by the boney vertebral endplate to which it is attached and is independent of the position of the other endplate.

17. The intervertebral implant of claim 1 wherein the core is adapted to provide relative movement of the endplates about a spine.

18. The intervertebral implant of claim 1, wherein the core includes an osteoinductive rigid matrix which provides for spinal fusion.

19. The intervertebral implant of claim 1, wherein the implant is an artificial disc.

20. The intervertebral implant of claim 1, wherein the implant is a fusion cage.

21. The implant of claim 1 wherein the conformable foam endplate is specifically designed to flex in a controlled fashion by manipulating its thickness, pore size, density and depth.

22. The endplate of claim 1 wherein the core is inflexible and the conformable endplate comprise a rubber-like material.

23. The endplate of claim 1 wherein the endplate has a dispensing inlet located on a sidewall, the inlet fluidly connected to an intra-endplate channel, which is fluidly connected to a dispensing orifice located on an outer surface of the prosthetic endplate.

24. The implant of claim 1 wherein the conformable foam endplate is filled with cement.

25. The implant of claim 1 wherein the conformable foam endplate is custom made from a CT scan.

26. The implant of claim 1 wherein the conformable foam endplate has a pocket for core insertion therein.

27. The implant of claim 1 wherein the conformable foam endplate comprises a high density edge section and a low density central section.

28. The implant of claim 1 wherein the conformable foam endplate comprises attachments thereto.

29. A kit, comprising: (a) at least two first conformable foam endplates, each first conformable foam endplate being conformable to a boney vertebral endplate under an anatomical load, each first conformable foam endplate having at least one dimension that is distinct from another first conformable endplate of the kit; (b) at least two second endplates, each second endplate having at least one dimension that is distinct from another second endplate of the kit; and (c) at least one core, the core being dimensioned for implantation between a first conformable endplate and a second endplate in an intervertebral space that has been prepared for placement of the first conformable endplate, the second endplate and the core, wherein, upon implantation, the first conformable endplate partitions the core from a first boney vertebral endplate with which the first conformable endplate is in contact, whereby the core does not contact the first boney vertebral endplate.

30. The kit of claim 29, wherein the second endplate is a conformable foam endplate and and is conformable to a second boney vertebral endplate under an anatomical load.

31. The kit of claim 30, wherein the second endplate, upon implantation of the second endplate and the core into an intervertebral space that has been prepared for placement of the first conformable endplate, the core and the second endplate, partitions the core from the second boney vertebral endplate, whereby the core does not contact the second boney vertebral endplate.

32. An intervertebral implant, comprising: (a) two conformable foam endplates, each endplate including a material that conforms to a boney vertebral endplate under an anatomical load; and (b) a core between the endplates, the core supporting boney vertebral endplates between which the conformable endplates have been placed and wherein a position of each conformable endplate is controlled at least in part by the boney vertebral endplate to which it is attached and is independent of the position of the other endplate.

33. A method of replacing an intervertebral disc, comprising the steps of: (a) removing at least a portion of an intervertebral disc to form an intervertebral disc space; (b) implanting a first conformable foam endplate into the intervertebral disc space and in contact with a first boney vertebral endplate, the first conformable foam endplate being conformable to the first boney vertebral endplate under an anatomical load; (c) implanting a second endplate into the intervertebral disc space and in contact with a second boney vertebral endplate; and (d) implanting a core between the first conformable endplate and the second endplate, wherein the first conformable endplate partitions the core from the first boney vertebral endplate, whereby the core does not contact the first boney vertebral endplate.

34. The method of claim 33, wherein the second endplate is a conformable foam endplate and is conformable to the second vertebral endplate under an anatomical load.

35. The method of claim 34, wherein the second endplate implanted partitions the core from the second boney vertebral endplate, whereby the core does not contact the second boney vertebral endplate.

36. The method of claim 35, further including the step of implanting at least one rigid plate between the core and at least one of the first conformable endplate and the second endplate.

37. An intervertebral implant trial, comprising:
a) a first conformable foam prosthetic endplate conformable to a first boney vertebral endplate under an anatomical load;
b) a second prosthetic endplate; and
c) a core between the prosthetic endplates.

38. An intervertebral implant, comprising:
a) a comformable core having an upper surface and a lower surface,
b) a first plurality of beads embedded in the upper surface of the core, and
c) a second plurality of beads embedded in the lower surface of the core.
